(19) United States
(12) Patent Application Publication
(10) Pub. No.: US 2023/0390439 A1
(43) Pub. Date: Dec. 7, 2023

(54) Disinfection System

(71) Applicant: JDC Corporation, Tokyo (JP)

(72) Inventors: Tsukasa Baba, Tokyo (JP); Hidetoshi Morimoto, Tokyo (JP); Hiroshi Obata, Tokyo (JP); Masakazu Sekiguchi, Tokyo (JP)

(21) Appl. No.: 18/250,602

(22) PCT Filed: Oct. 1, 2021
(86) PCT No.: PCT/JP2021/036426

§ 371 Date: Apr. 26, 2023

Related U.S. Application Data
(60) Provisional application No. 63/120,241, filed on Dec. 2, 2020.

(51) Int. Cl. A61L 2/10; A61L 2/18; A61L 2/24

(52) U.S. Cl. CPC A61L 2/10; A61L 2/18; A61L 2/24; A61L 2202/14; A61L 2202/11; A61L 2202/25

(57) ABSTRACT

A disinfection system includes, to enable prevention of infection of infectious diseases, a first sensing device that senses entry of a person into a first room and a disinfection device including at least one of a first irradiation device that emits invisible light to a disinfection area or a first fluid supply device that supplies a fluid to the disinfection area. A first control device of the disinfection system operates at least one of the first irradiation device 3 or the first fluid supply device 5 when the first sensing device 2 has sensed the entry of a person into the first room 1.

### TECHNICAL FIELD

The present invention relates to a disinfection system capable of disinfecting, for example, viruses.

### BACKGROUND

JP Patent Publication No. 2018-117708 A discloses a cough detection device that is provided with a motion detection unit and a sound detection unit corresponding to a seat in a living space of an office or a classroom or in a moving body such as a bus, a train, or an airplane, and detects at which seat a person has coughed. Further, JP Patent Publication No. 2018-117708 A discloses that an infectious disease is detected on the basis of a detection result of the cough detection device.

### SUMMARY

However, JP Patent Publication No. 2018-117708 A limits the disclosure to the detection of an infectious disease and does not disclose measures such as prevention of infection of the infectious disease.

Therefore, an object of the present invention is to provide a disinfection system (also called a disinfection apparatus) capable of preventing infection of infectious diseases.

A first disinfection system according to an embodiment of the present invention includes: a first sensing device configured to sense entry of a person into a first room; a disinfection device including at least one of a first irradiation device that emits invisible light to a disinfection area or a first fluid supply device that supplies a fluid to the disinfection area; and a first control device that operates at least one of the first irradiation device or the first fluid supply device when the sensing device has sensed the entry of a person into the first room.

A second disinfection system according to an embodiment of the present invention includes: a second sensing device configured to sense entry into a second room; a detection device configured to detect a cough or a sneeze of a person who stays in the second room; a disinfection device including at least one of a second irradiation device that emits invisible light to the second room or a second fluid supply device that supplies a fluid to the second room; and a second control device configured to drive the disinfection device when the detection device has detected the cough or the sneeze.

A third disinfection system according to an embodiment of the present invention includes: a first disinfection device provided in a first room and configured to disinfect the first room; a second disinfection device provided in a second room different from the first room and configured to disinfect the second room; and a control device configured to operate the first disinfection device and the second disinfection device under different conditions.

According to the first disinfection system, when the first sensing device senses the entry of a person into the first room, the first control device operates at least one of the first irradiation device or the first fluid supply device. Therefore, it is possible to efficiently disinfect the disinfection area.

According to the second disinfection system, the second control device operates the second disinfection device when the second sensing device senses entry of a person into the second room or when the detection device detects a cough or a sneeze of the person in the second room. Therefore, it is possible to efficiently perform disinfection in response to sensing the entry of a person into the second room or detecting the cough or sneeze in the second room.

According to the third disinfection system, the control device operates the first disinfection device and the second disinfection device under different conditions. Therefore, it is possible to perform harmless and efficient disinfection for human bodies.

### BRIEF DESCRIPTION OF THE DRAWINGS

FIG. 1 is a schematic view illustrating a first room and a second room according to an embodiment of the present invention.

FIG. 2 is a schematic view (cross-sectional view) illustrating the second room according to an embodiment of the present invention.

FIG. 3 is a block diagram of a main part of the first room according to an embodiment of the present invention.

FIG. 4 is a block diagram of a main part of the second room according to an embodiment of the present invention.

FIG. 5 is a diagram illustrating a flowchart executed by a control device of the first room according to an embodiment of the present invention.

FIG. 6 is a diagram illustrating a flowchart executed by a control device of the second room according to the embodiment of the present invention.

### DETAILED DESCRIPTION

Hereinafter, embodiments of the present invention will be described in detail with reference to the accompanying drawings. Note that the present invention is not limited by the embodiments to be described below.

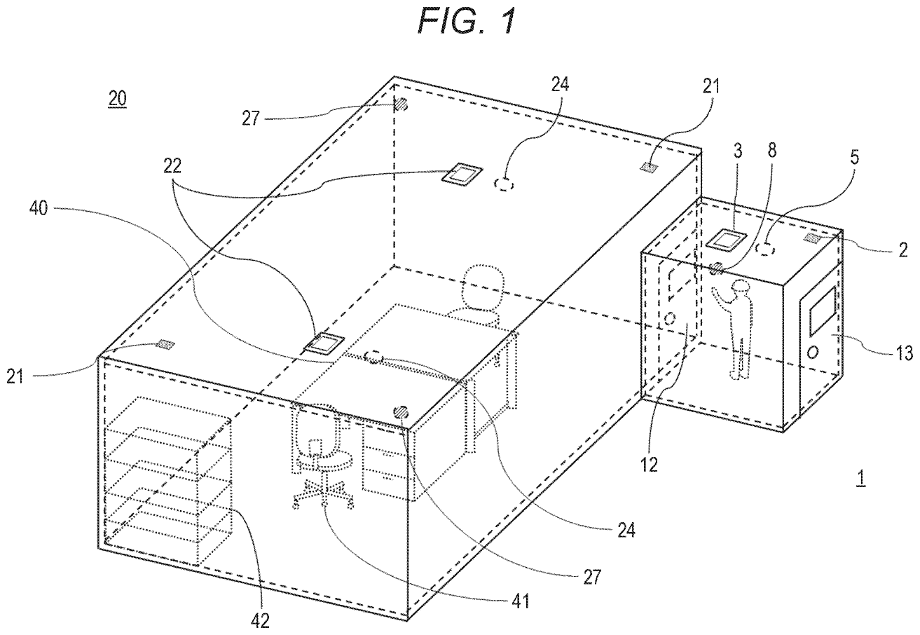

FIG. 1 is a schematic view illustrating a first room 1 and a second room 20 of the present embodiment. As illustrated in FIG. 1, the first room 1 is connected to the second room 20, and entry to and exit from the second room 20 are performed from the first room 1 using a doorway 12. Further, the first room 1 has a doorway 13 separate from the doorway 12 on a side of the second room 20. In the present embodiment, the second room 20 is a temporary office or a break room installed at a construction site or the like, and the first room 1 is installed at a doorway of the second room 20 and is a room where disinfection is conducted before entry to the second room 20.

As the second room 20, temporary offices and break rooms having various sizes are installed depending on a scale of the construction site. Therefore, a size of the first room 1 is changed according to the second room 20 and is also intended for disinfection before entry to the second room 20. The first room 1 is thus smaller than the second room 20.

The first room 1 includes a first sensing device 2 that senses entry of a person, a first irradiation device 3 that emits invisible light to an inside of the first room 1, a first fluid supply device 5 that supplies a fluid into the first room 1, and a first imaging device 8.

The first sensing device 2 senses entry of a person into the first room 1. For example, a human sensor that detects presence of a person by infrared rays, ultrasonic waves, or visible light can be used. The first sensing device 2 may be installed on, for example, an upper part of the first room 1 (for example, on a ceiling or near the ceiling) or a plurality of the first sensing devices 2 may be installed near the doorways of the first room 1 (at a plurality of locations of the doorway 12 and the doorway 13) according to the size of the first room 1.

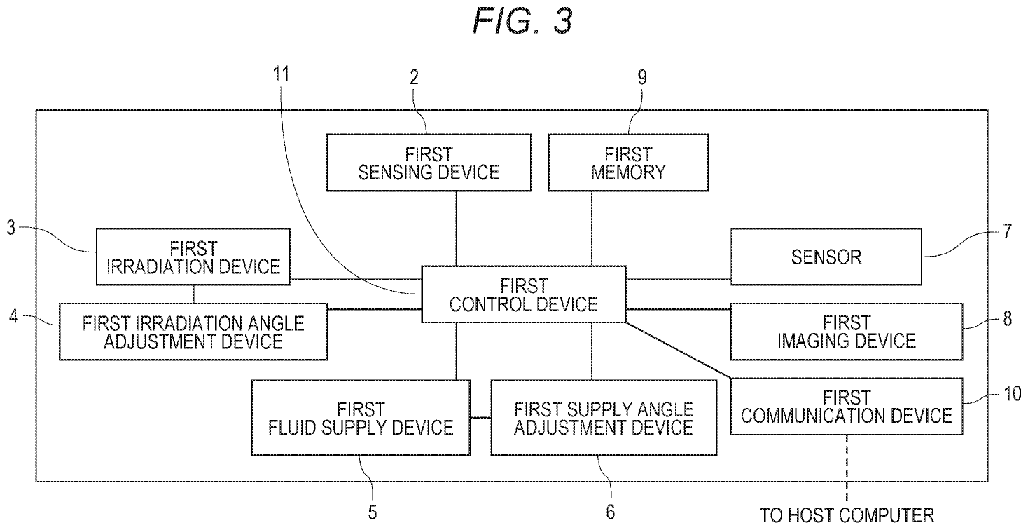

A thermometer may be installed as a sensor 7 (see FIG. 3) near the side of the doorway 12 between the first room 1 and the second room 20, and timer equipment may also be installed. As the thermometer, a non-contact-type thermometer is favorable, and for example, a thermometer using infrared rays can be used. However, the embodiments are not limited thereto. In this case, to enable measurement of temperature before entry to the second room 20 from the first room 1, it is favorable that the thermometer is installed on a side wall or the like near the doorway 12, and measurement is performed while a person faces the thermometer before entering the second room 20.

A camera or the like may be installed as the first imaging device 8 near the side of the doorway 12 between the first room 1 and the second room 20. It is favorable to specify a person who has entered or exited from the room from a still image, a moving image, or the like captured by the first imaging device 8, and to record the time when the person has entered or exited from the first room 1 or the like, using timer equipment built in the first imaging device 8 or separately provided. From these records, it is possible to specify a person who has stayed in the first room 1 and the second room 20 and a time period of the stay.

The first irradiation device 3 is provided on an upper part of the first room 1 (for example, on the ceiling or near the ceiling), and emits invisible light such as ultraviolet rays downward from the upper part of the first room 1. As the ultraviolet rays emitted by the first irradiation device 3, far ultraviolet C waves or rays having a wavelength of 205 to 230 nm are emitted. This is because the far ultraviolet C waves having the wavelength of 205 to 230 nm do not reach human cells and thus do not harm the human body, but the waves penetrate bacteria and viruses in the air and on a surface of an object and can disinfect the air and the surface of the object. By adopting the far ultraviolet C waves having the wavelength of 205 to 230 nm, the invisible light can be safely emitted from the first irradiation device 3 even in the case where a person stays in the first room 1. Hereinafter, in the present embodiment, disinfection that is harmless to a person is referred to as first disinfection.

In a case where no person stays in the first room 1, the ultraviolet rays emitted by the first irradiation device 3 may be deep ultraviolet rays (UV-C) having a wavelength of 100 to 280 nm. Because the deep ultraviolet rays (UV-C) exhibit a stronger sterilizing action than the far ultraviolet C waves having the wavelength of 205 to 230 nm, it is possible to disinfect the inside of the first room 1 more reliably. However, the disinfection is performed only when no person stays in the first room 1 because DNA damage is induced to human cells as well and is harmful to a person. Hereinafter, in the present embodiment, the disinfection that can be stronger but is harmful to a person is referred to as second disinfection.

The ultraviolet rays emitted by the first irradiation device 3 include two or more types including the far ultraviolet C waves having the wavelength of 205 to 230 nm that does not affect the human body and the deep ultraviolet rays (UV-C) having the wavelength of 100 to 280 nm that can be expected to have a stronger sterilization effect but are harmful to a person. Therefore, as the first irradiation device 3, two or more types of irradiation devices may be used as the first irradiation device 3, including an irradiation device that emits the far ultraviolet C waves having the wavelength of 205 to 230 nm and an irradiation device that emits the deep ultraviolet rays (UV-C) having the wavelength of 100 to 280 nm, or an irradiation device having a wavelength switching function may be used as the first irradiation device 3.

The disinfection by invisible light of the first irradiation device 3 is not limited to the person who enters the first room 1. Instead, disinfection is performed for the entire inside of the first room 1. The disinfection is performed for a floor, a wall, a doorknob, a handle, and the like as a place where the person who has entered the first room 1 comes into contact or a place where viruses or bacteria attached to the human body are likely to spread and adhere. To emit the invisible light to such an object to be disinfected from the first irradiation device 3, a plurality of the first irradiation devices 3 may be provided, or the first irradiation device 3 may be provided on the floor or the wall. Further, a first irradiation angle adjustment device 4 (see FIG. 3) may be provided and adjust an irradiation angle of the first irradiation device 3.

The first fluid supply device 5 is provided on an upper part of the first room 1 (for example, on the ceiling or near the ceiling), and sprays a fluid downward from the upper part of the first room 1. An example of the first fluid supply device 5 is to supply a disinfectant liquid to a disinfection area. As the disinfectant liquid, various types of liquids such as an alcohol-based solution, an aldehyde-based solution, a chlorine-based solution, an iodine-based solution, an oxidant-based solution, and a quaternary ammonium salt-based solution can be used. In the present embodiment, in a case where a person stays in the first room 1, an alcohol-based disinfectant solution is used (first disinfection). In a case where no person stays in the first room 1, a non-alcohol-based disinfectant solution may be used (second disinfection). In this way, the person staying in the first room 1 is prevented from being harmed. In this case, a plurality of containers may be provided in the first fluid supply device 5, the alcohol-based disinfectant solution may be placed in one container, and the non-alcohol-based disinfectant solution may be placed in another container. Note that the number of containers may be three or more, and the number of types of disinfectant solutions to be supplied may be two or more. Further, to disinfect the disinfection area inside the first room 1, a plurality of the first fluid supply devices 5 may be provided, or the first fluid supply devices 5 may be provided on the floor or the wall. Further, a first supply angle adjustment device 6 (see FIG. 3) may be provided and adjusts a supply angle of the first fluid supply device 5.

The first fluid supply device 5 may supply compressed air as a fluid. In this case, the first fluid supply device 5 may be installed on an upper part of the doorway 12 or near the doorway 13 (for example, on the ceiling or near the ceiling) and removes bacteria or viruses adhering to clothes or the like of a person entering the first room 1. Further, an air shower may be formed so that the air in the first room 1 does not flow into the second room 20.

Hereinafter, device configurations used in the first room 1 will be described using the block diagram of FIG. 3. A first memory 9 is a non-volatile memory (for example, a flash memory) that stores a disinfection history of the first room 1 described above (an irradiation history of the first irradiation device 3 and a supply history of the first fluid supply device 5), data of the face of a person who enters and leaves the first room 1, data measured by the thermometer, the time, and the like.

A first communication device 10 is a wireless communication unit that accesses a wide area network such as a host computer or the Internet. In the present embodiment, the first communication device 10 transmits a sensing result of the first sensing device 2, the irradiation history of the first irradiation device 3, the supply history of the first fluid supply device 5, and the like to the host computer.

The first memory 9 may store the sensing result of the first sensing device 2, the irradiation history of the first irradiation device 3, the supply history of the first fluid supply device 5, and the like. Furthermore, the first memory 9 may store an imaging record by the first imaging device 8 and the time of the imaging record, a detection result by the sensor 7, and the like.

A first control device 11 includes a CPU and performs control for disinfecting bacteria and viruses in the first room 1. Note that the control for disinfecting the bacteria and viruses in the first room 1 by the first control device 11 will be described below.

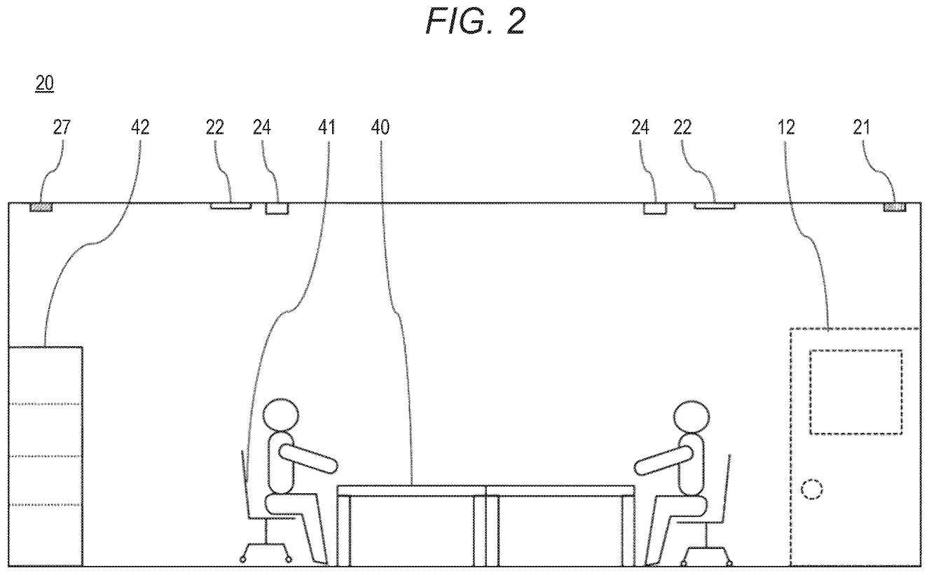

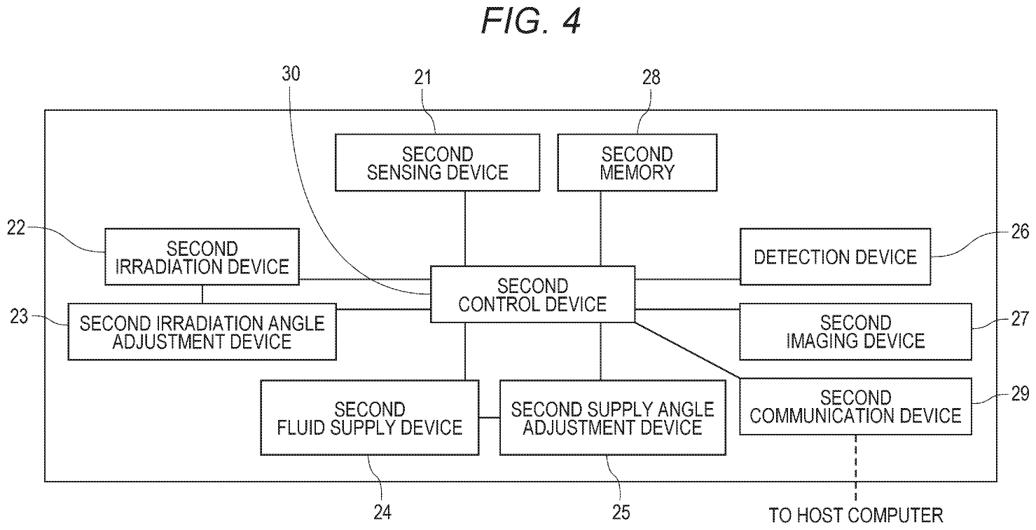

FIG. 2 is a schematic view illustrating an interior of the second room 20. The second room 20 is a temporary office or a break room at a construction site, and includes furniture and fixtures such as a desk 40, a chair 41, and a shelf 42, a second sensing device 21 that senses entry and stay in the room of a person, a second irradiation device 22 that emits invisible light to the inside of the second room 20, a second fluid supply device 24 that supplies a fluid into the second room 20, a detection device 26 (see FIG. 4) that detects a cough or a sneeze of the person in the room, and a second imaging device 27. Note that a layout of the furniture and fixtures is an example, and various changes can be made.

The second sensing device 21 senses entry of a person into the second room 20. For example, a human sensor that detects presence of a person by infrared rays, ultrasonic waves, or visible light can be used, as in the first sensing device 2. The second sensing device 21 is installed on an upper part of the second room 20 (a ceiling, a wall near the ceiling, and the like), for example. In FIG. 2, the second sensing devices 21 are installed at two diagonal locations among ceiling corners of the second room 20, but the second sensing devices 21 may be installed at one location near a center of the ceiling or three or more locations. The second sensing devices 21 are installed according to the size of the second room 20.

Furthermore, a camera or the like may be installed as the second imaging device 27 at a place (for example, the ceiling or the wall near the ceiling) where the camera can capture the entire inside of the second room 20. In the case where the disinfection is performed when no person stays in the second room 20 (second disinfection), behavior of a person who has entered the second room 20 can be recognized from a photograph, a moving image, or the like captured by the second imaging device 27, and the disinfection area can be determined on the basis of the data.

The detection device 26 is a sound detection unit such as a microphone as described in JP Patent Publication No. 2018-117708 A, and the detection device 26 detects a cough or a sneeze of the person who stays in the room. Whether a sound signal detected by the detection device 26 is a cough or a sneeze can be detected by a frequency analysis for the sound signal on the basis of an output of the detection device 26. In this case, the sound signal detected by the detection device 26 may be wirelessly transmitted to the host computer, and the frequency analysis may be performed by the host computer or by a second control device 30 (see FIG. 4).

As the detection device 26, a motion detection unit having an acceleration sensor as described in JP Patent Publication No. 2018-117708 A may be provided to detect the cough or sneeze of the person who stays in the room. A numerical value or a pattern of acceleration of when the person coughs or sneezes is stored as a reference in a memory of the host computer or a second memory 28 (see FIG. 4), and whether the person staying in the room has coughed or sneezed can be detected by a comparison with a result detected by the acceleration sensor.

By providing both the microphone and the acceleration sensor as the detection device 26, it is possible to accurately detect the cough or sneeze of the person staying in the room. Note that the numbers of microphones and acceleration sensors may be one or plural, and an arrangement place thereof can be arbitrarily set, but it is favorable to provide the microphone and the acceleration sensor on the desk 40 or the like so as to face a seat of the person staying in the room in order to detect the cough or sneeze in the seat.

It is conceivable that the second room 20 has a certain size and multiple people stay in the room. Therefore, by providing a plurality of the detection devices 26 on the desks 40 of respective seats, it is possible to specify in which area a person who has coughed or sneezed is present among people who stay in the room, and it is possible to disinfect the area where the cough or sneeze has been detected in a pinpointed manner. Further, to suppress a cost related to the detection device 26, a device capable of detecting the loudness of a sound, such as a noise meter, may be installed at a plurality of locations (for example, on the wall side), and an area near the device that has detected a larger sound may be specified as the area where the cough or sneeze has occurred.

When at least one of the second irradiation device 22 or the second fluid supply device 24 (e.g., either or both) is driven at the timing when the detection device 26 detects the cough or sneeze of the person staying in the room, the bacteria or viruses can be immediately neutralized even in a case where the bacteria or viruses are included in the cough or sneeze.

The second irradiation device 22 is provided on an upper part of the second room 20 (for example, on a ceiling or near the ceiling), and emits invisible light such as ultraviolet rays downward from the upper part of the second room 20. In the present embodiment, the second irradiation device 22 emits the far ultraviolet C waves having the wavelength of 205 to 230 nm as ultraviolet rays, as in the first irradiation device 3. This is because it is considered that the far ultraviolet C waves having a wavelength of 205 to 230 nm do not reach human cells and thus do not harm the human body, but the waves penetrate bacteria and viruses in the air and on a surface of an object and can disinfect the air and the surface of the object. By adopting the far ultraviolet C waves having the wavelength of 205 to 230 nm, the invisible light can be safely emitted from the second irradiation device 22 even in the case where a person enters in the second room 20 (first disinfection).

The second irradiation device 22 emits the far ultraviolet C waves to a door knob and a handle of the doorway 12 and the furniture and fixtures such as the desk 40, the chair 41, and the shelf 42, as an area where the person who has entered the second room 20 comes into contact, and an area where a cough or a sneeze may be scattered and attached when the person who has entered the second room 20 has coughed or sneezed. To emit the far ultraviolet C waves to such disinfection areas, a plurality of the second irradiation devices 22 may be provided, or the second irradiation device 22 may be provided on the floor side or the wall. Further, a second irradiation angle adjustment device 23 may be provided to adjust an irradiation angle of the second irradiation device 22. It is also possible to disinfect the bacteria and viruses floating in the second room 20 by the emission of the far ultraviolet C waves by the second irradiation device 22.

Further, when the stay of a person in the second room 20 is not sensed, the second irradiation device 22 may emit the deep ultraviolet rays (UV-C) having the wavelength of 100 to 280 nm from the second irradiation device 22 to the door knob and the hand of the doorway 12, the furniture and fixtures such as the desk 40, the chair 41, and the shelf 42, the floor, and the wall, as the place may be touched by a hand of the person who has stayed in the room, or the place where the cough or sneeze may be scattered and attached when the person who has stayed in the room has coughed or sneezed (second disinfection). The deep ultraviolet rays (UV-C) exhibit a stronger sterilizing action than the far ultraviolet C waves having the wavelength of 205 to 230 nm, but the rays induce DNA damage on human cells as well and may be harmful to a person. Therefore, in the case where the stay of a person in the second room 20 is not detected, the deep ultraviolet rays (UV-C) are emitted from the second irradiation device 22. Note that, in a case where a person enters the second room 20 when the deep ultraviolet rays (UV-C) are emitted from the second irradiation device 22, the emission of the deep ultraviolet rays (UV-C) is immediately stopped.

The second fluid supply device 24 is provided on an upper part of the second room 20 (for example, on the ceiling or near the ceiling), and sprays a fluid downward from the upper part of the second room 20. An example of the second fluid supply device 24 supplies a disinfectant liquid to the furniture and fixtures such as the desk 40, the chair 41, and the shelf 42, the floor, and the wall, as the place may be touched by a hand of the person who has stayed in the room, or the place where the cough or sneeze may be scattered and attached when the person who has stayed in the room has coughed or sneezed. As the disinfectant liquid, various types of liquids such as an alcohol-based solution, an aldehyde-based solution, a chlorine-based solution, an iodine-based solution, an oxidant-based solution, and a quaternary ammonium salt-based solution can be used, as in the first fluid supply device 5. In the present embodiment, in a case where a person stays in the second room 20, an alcohol-based disinfectant solution is used (first disinfection). In a case where no person stays in the disinfection room, a non-alcohol-based disinfectant solution may be used (second disinfection). In this way, the person staying in the second room 20 is prevented from being harmed. In this case, a plurality of containers may be provided in the second fluid supply device 24, the alcohol-based disinfectant solution may be placed in one container, and the non-alcohol-based disinfectant solution may be placed in another container. Note that the number of containers may be three or more, and the number of types of disinfectant solutions to be supplied may be two or more. Further, to disinfect the disinfection area inside the second room 20 a plurality of the fluid supply devices may be provided, or the fluid supply devices may be provided on the floor side or the wall.

The second fluid supply device 24 may supply compressed air as a fluid. In this case, the second fluid supply device 24 may be installed on an upper part of the doorway 12 (for example, on the ceiling or near the ceiling) of the second room 20 and removes bacteria or viruses adhering to clothes or the like of a person entering the second room 20. Further, an air shower may be formed so that the air in the first room 1 does not flow into the second room 20.

Hereinafter, device configurations used in the second room 20 will be described using the block diagram of FIG. 4. The second memory 28 is a non-volatile memory (for example, a flash memory) that stores a numerical pattern of the above-described acceleration, a disinfection history of the second room 20 (an irradiation history of the second irradiation device 22 and a supply history of the second fluid supply device 24), data of the face of a person who enters and leaves the second room 20, a range of behavior of the person who has entered the second room 20, and the like.

A second communication device 29 is a wireless communication unit that accesses a wide area network such as a host computer or the Internet. In the present embodiment, the second communication device 29 transmits a sensing result of the second sensing device 21, the irradiation history of the second irradiation device 22, the supply history of the second fluid supply device 24, and the like to the host computer.

The second memory 28 may store the sensing result of the second sensing device 21, the irradiation history of the second irradiation device 22, the supply history of the second fluid supply device 24, and the like. Furthermore, the second memory 28 may store an imaging record by the second imaging device 27, and the like.

The second control device 30 includes a CPU and performs control for disinfecting bacteria and viruses in the second room 20. Note that the control for disinfecting the bacteria and viruses in the second room 20 by the second control device 30 will be described below.

### Description of Flowchart

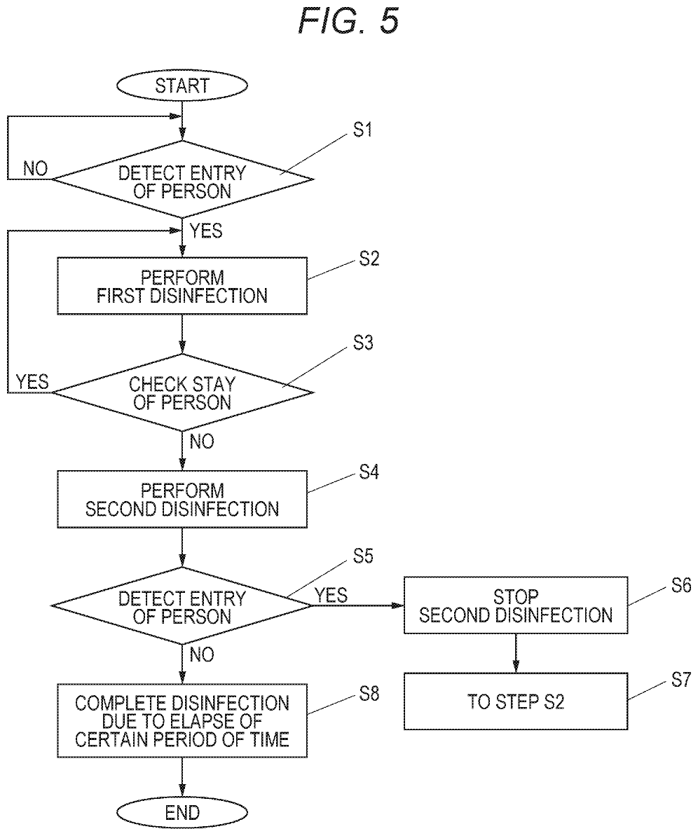

The control for disinfecting the bacteria and viruses in the first room 1 by the control device of the present embodiment configured as described above will be continuously described with reference to the flowchart of FIG. 5. Note that the present flowchart is assumed to be performed from when a person enters the first room 1. Note that, as the entry into the first room 1, entry from the doorway 12 connected to the second room 20 (exit from the second room 20) and entry from the doorway 13 different from the second room 20 side are conceivable. This time, the description will be given on the assumption that entry into the first room 1 is made from the doorway 13.

The first control device 11 performs detection by the first sensing device 2 and detects whether a person has entered the first room 1 (step S1).

In a case of detecting that a person has entered the first room 1, the first control device 11 performs the first disinfection (disinfection harmless to the human body) by the first irradiation device 3 and the first fluid supply device 5 (step S2). Because the person stays in the room, the first irradiation device 3 emits the far ultraviolet C waves having the wavelength of 205 to 230 nm, favorably around 222 nm, which does not affect the human body. An irradiation time of the far ultraviolet C waves by the first irradiation device 3 can be set to several seconds to several minutes according to intensity of the far ultraviolet C waves. The first fluid supply device 5 performs disinfection using an alcohol-based disinfectant solution that does not affect the human body.

Note that, in step S2, when the first control device 11 detects the entry of a person, the body temperature may be measured by the thermometer described above. Because it is conceivable that a plurality of persons enters the room, a face recognition device may be provided near the thermometer installation location and recognize the face of the person entering the room and associate the person entering the room with a measured body temperature and the time. In this case, face data of the person entering the room may be registered in the first memory 9.

The first control device 11 checks whether a person stays in the first room 1 or not by the first sensing device 2 (step S3). Here, in a case where a person stays in the first room 1, the first disinfection is continuously performed (step S2). In a case where the person has left the first room 1 and no person stays in the first room 1, the second disinfection is performed by the first irradiation device 3 and the first fluid supply device 5 (step S4). This is because the first room 1 is a room in which disinfection is performed before entry to the second room 20, and a stay time of a person in the first room 1 is short. Sufficient disinfection may not be able to be performed by the disinfection in step S2 that is the first disinfection. Therefore, the disinfection is performed even after the person has left the first room 1. Here, the processing proceeds to step S4 on the assumption that the person has left the first room 1.

In the case where the person has left the first room 1, the first control device 11 emits the deep ultraviolet rays (UV-C) having the wavelength of 100 to 280 nm having a stronger sterilization effect from the first irradiation device 3 as the second disinfection. The irradiation time of the deep ultraviolet rays (UV-C) by the first irradiation device 3 can be set between several seconds and several minutes according to the intensity of the deep ultraviolet rays (UV-C). Further, in the disinfection by the first fluid supply device 5, a non-alcohol-based disinfectant solution can be used. Note that there is a possibility that a person enters the first room 1 when the second disinfection is performed by the first irradiation device 3 or the first fluid supply device 5. In such a situation, it is necessary to sense and feed back the presence or absence of the person entering the first room 1 (step S5). Here, the processing proceeds to step S6 on the assumption that a person has entered the first room 1 during the execution of the second disinfection.

In the case of sensing that a person has entered the first room 1 during the execution of the second disinfection, the first control device 11 immediately stops the second disinfection by the first irradiation device 3 and the first fluid supply device 5 (step S6), returns the processing to step S2 (step S7), switches the processing to the execution of the first disinfection that does not affect the human body, and repeats steps S3 to S5 described above. Here, in step S5, the description will be continued assuming that there is no person entering the first room 1 during the execution of the second disinfection.

In step S5, in the case where no person has entered the first room 1 during the execution of the second disinfection, the first control device 11 completes the second disinfection due to elapse of a certain period of time for the operation of the first irradiation device 3 and the first fluid supply device 5 (step S8), and completes all the operations.

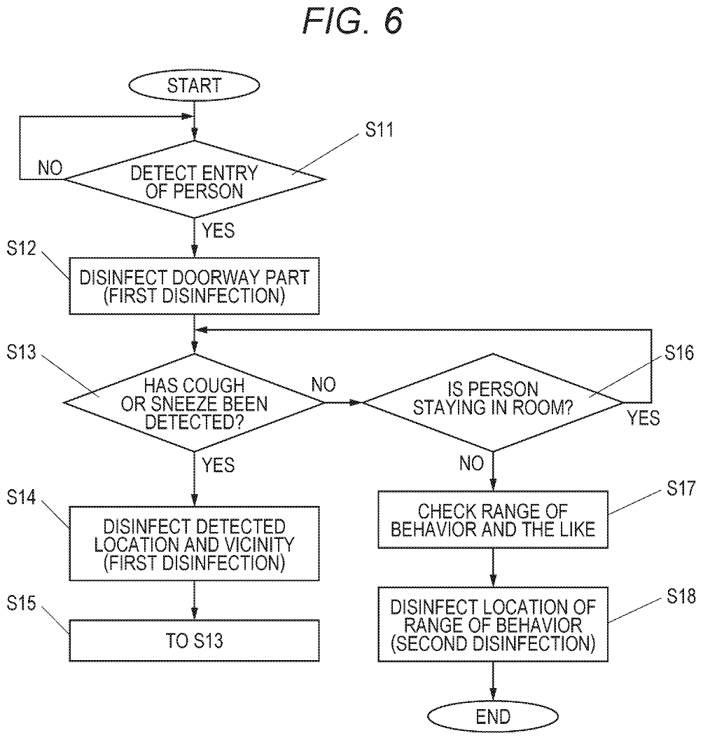

Next, the control for disinfecting the bacteria and viruses in the second room 20 by the control device of the present embodiment will be continuously described with reference to the flowchart of FIG. 6. Note that the present flowchart is assumed to be performed from when a person has entered the second room 20.

The second control device 30 performs detection by the second sensing device 21 and detects whether a person has entered the second room 20 (step S11). Here, the processing proceeds to step S12 on the assumption that the person has entered the second room 20.

In a case where the second control device 30 has sensed the entry of a person to the second room 20, the second control device 30 drives at least one of the second irradiation device 22 or the second fluid supply device 24 and performs the disinfection (first disinfection) of the vicinity of the doorway 12 (the doorknob, the handle of door, and the like) (step S12). In the first disinfection, because the person stays in the room, emission of the far ultraviolet C wave having the wavelength of 205 to 230 nm, favorably, the wavelength around 222 nm, is performed in the disinfection area by the second irradiation device 22, and the disinfection using an alcohol-based disinfectant solution is performed by the second fluid supply device 24.

Next, in the state where the person stays in the second room 20, the second control device 30 causes the detection device 26 to detect whether the person in the room has coughed or sneezed (step S13), and determines whether or not to perform disinfection on the basis of the detection result. Here, the processing proceeds to step S14 on the assumption that the cough or sneeze of the person staying in the room has been detected.

The second control device 30 drives at least one of the second irradiation device 22 or the second fluid supply device 24 and performs the disinfection (first disinfection) around a location where the cough or sneeze has been detected (step S14). In the disinfection when the cough or sneeze has been detected, because the person stays in the room, the second control device 30 performs emission of the far ultraviolet C wave having the wavelength of 205 to 230 nm, favorably, the wavelength around 222 nm, in the disinfection by the second irradiation device 22, and performs the disinfection using an alcohol-based disinfectant solution by the second fluid supply device 24.

As the cough or sneeze of the person staying in the second room 20, there is a possibility that the same person continuously coughs or sneezes, or there is a possibility that a plurality of persons staying in the room simultaneously or continuously coughs or sneezes. Therefore, the second control device 30 may drive both the second irradiation device 22 and the second fluid supply device 24 when detecting a continuous cough or sneeze. As an object to be disinfected by the second irradiation device 22 and the second fluid supply device 24, the second control device 30 may cause the second irradiation device 22 to perform the disinfection for the person who has coughed or sneezed or a periphery of the person, and cause the second fluid supply device 24 to perform the disinfection for the furniture and fixtures such as the desk. Further, in the case where the second control device 30 continuously detects the cough or sneeze, an irradiation time of the invisible light by the second irradiation device 22 may be lengthened, or a spraying time of the fluid from the second fluid supply device 24 may be lengthened (a spraying amount may be increased).

After performing the first disinfection in step S14, the second control device 30 returns to step S13 (step S15), and subsequently detects whether the person staying the second room 20 has coughed or sneezed (step S13). Here, the processing proceeds to step S16 on the assumption that the cough or sneeze of the person staying in the second room 20 has not been detected.

The second control device 30 checks the presence or absence of a person who stays in the second room 20 by the second sensing device 21. In the case where a person stays in the second room 20, the second control device 30 repeats steps S13 to S16 described above. Here, the processing proceeds to step S17 on the assumption that the person has left the second room 20 (no person staying in the second room 20).

The second control device 30 causes the second imaging device 27 installed in the second room 20 to capture the range of behavior of the person staying in the room and the second memory 28 to store the range of behavior. The second control device 30 reads and checks information recorded in the second memory 28 for the range of behavior of the person staying in the second room 20 (step S17).

The second control device 30 drives at least one of the second irradiation device 22 or the second fluid supply device 24 and performs the second disinfection on the basis of the range of behavior of the person staying in the second room 20 read from the second memory 28 (step S18). In the second disinfection, since no person stays in the room, the second irradiation device 22 can perform the disinfection by emitting the deep ultraviolet rays (UV-C) having the wavelength of 100 to 280 nm exhibiting a strong sterilizing action. In the second fluid supply device 24, disinfection using a non-alcohol-based disinfectant solution can be performed.

The effect of disinfection by the second irradiation device 22 (the effect of ultraviolet sterilization) is determined by integration (integrated light amount or ultraviolet irradiation amount) of the intensity of light (illuminance) and the time to apply light (irradiation time). Therefore, the irradiation time by the second irradiation device 22 depends on the illuminance used for the second irradiation device 22, but the time about several minutes to several tens of minutes is necessary. When the disinfection in the range of behavior of the person staying in the indoor space is completed, the flowchart ends.

The disinfection by the first irradiation device 3 or the second irradiation device 22 (hereinafter, irradiation device) and the disinfection by the first fluid supply device 5 or the second fluid supply device 24 (hereinafter, fluid supply device) are not visually recognizable by a person. Therefore, it is desirable to enable whether or not disinfection is being performed to be recognizable. For example, in the case where the disinfection by the irradiation device is being performed, a disinfection status can be clearly indicated to a person by a visual indicator, such as by installing a monitor that displays a type of irradiation or turning on a lamp or the like of a predetermined color for each wavelength of irradiation (depending on whether the wavelength is harmful to the human body). Moreover, in the case where the disinfection using the deep ultraviolet rays (UV-C) harmful to the human body is being performed, it is desirable to cause a person to easily recognize that entry to the room is dangerous by sounding a warning sound, and it is favorable to play the warning sound before performing irradiation.

The above-described embodiments are merely examples for describing the present invention, and various modifications can be made without departing from the scope of the present invention. The second room 20 is a temporary office such as a construction site or a break room, but the second room 20 can be implemented as various other buildings such as a school, a hospital, an office, or a commercial facility.

Further, by recording persons who have entered and left the first room 1 and the second room 20 and the time of their entry and exit in the first memory 9 and the second memory 28, it is also possible to find a person who has come into very-close contact with an infected person when occurrence of the infected person is confirmed among the persons who have entered and left the rooms.

The following is a list of reference signs used in the drawing figures and in this specification.
1 First room (disinfection room)
2 First sensing device
3 First irradiation device
4 First irradiation angle adjustment device
5 First fluid supply device
6 First fluid supply angle adjustment device
7 Sensor
8 First imaging device
9 First memory
10 First communication device
11 First control device
12 Doorway (between first room and second room)
13 Doorway (outside)
20 Second room
21 Second sensing device
22 Second irradiation device
23 Second irradiation angle adjustment device
24 Second fluid supply device
25 Second fluid supply angle adjustment device
26 Detection device
27 Second imaging device
28 Second memory
29 Second communication device
30 Second control device
40 Desk
41 Chair
42 Shelf

1.-10. (canceled)

11. A disinfection apparatus comprising:
a sensing device configured to sense a presence or an absence of a person in a disinfection area;
an irradiation device capable of irradiating the disinfection area with invisible light of a first wavelength and with invisible light of a second wavelength different from the first wavelength;
a controller that causes the irradiation device to irradiate with the first wavelength when the sensing device detects the presence of the person in the disinfection area, and causes the irradiation device to irradiate with the second wavelength when the sensing device does not sense the person in the disinfection area; and
a visual indicator that displays information in connection with the irradiation of the irradiation device.

12. The disinfection apparatus of claim 11, wherein
the visual indicator displays according to the first and second wavelengths.

13. The disinfection apparatus of claim 11, wherein
when the sensing device detects a person while the irradiation device is irradiating with the second wavelength, the controller stops irradiation with the second wavelength by the irradiation device.

14. The disinfection apparatus of claim 11, wherein
the controller sets an irradiation time for irradiating with the first wavelength.

15. The disinfection apparatus of claim 11, wherein
the irradiation device irradiates ultraviolet light with a wavelength of around 222 nm as the first wavelength.

16. The disinfection apparatus of claim 11, wherein
the irradiation device irradiates a fixture in the disinfection area by the first wavelength.

17. The disinfection apparatus of claim 11, wherein
the irradiation device irradiates a fixture in the disinfection area by the second wavelength.

18. A disinfection apparatus comprising:
a sensing device configured to sense a presence or an absence of a person in a disinfection area;
an irradiation device capable of irradiating the disinfection area with invisible light of a first wavelength and with invisible light of a second wavelength different from the first wavelength; and
a controller that causes the irradiation device to irradiate with the first wavelength when the sensing device detects the presence of the person in the disinfection area, causes the irradiation device to irradiate with the second wavelength when the sensing device does not sense the person in the disinfection area, and terminates irradiation after a period of time.

19. The disinfection apparatus of claim 18, wherein
when the sensing device detects the person while the irradiation device is irradiating with the second wavelength, the controller stops irradiation with the second wavelength by the irradiation device.

20. The disinfection apparatus of claim 18, wherein
the controller sets an irradiation time for irradiating with the first wavelength.

21. The disinfection apparatus of claim 18, wherein
the irradiation device irradiates ultraviolet light with a wavelength of around 222 nm as the first wavelength.

22. The disinfection apparatus of claim 18, wherein
the irradiation device irradiates a fixture in the disinfection area by the first wavelength.

23. The disinfection apparatus of claim 18, wherein
the irradiation device irradiates a fixture in the disinfection area by the second wavelength.

24. A disinfection method for disinfecting a disinfection area, the method comprising:
sensing a presence or an absence of a person in the disinfection area;
irradiating with a first wavelength when the presence of the person in the disinfection area is detected, and irradiate with a second wavelength different from the first wavelength when the absence of the person in the disinfection area is detected; and
displaying information in connection with the irradiation.

25. The disinfection method of claim 24, wherein
the displaying displays according to the first and second wavelengths.

26. The disinfection method of claim 24, wherein
when the person is detected during irradiation with the second wavelength, irradiation with the second wavelength is stopped.

27. The disinfection method of claim 24, comprising:
setting an irradiation time for irradiating with the first wavelength.

28. The disinfection method of claim 24, wherein
the irradiating comprises irradiating ultraviolet light with a wavelength of around 222 nm as the first wavelength.

29. The disinfection method of claim 24, wherein
the irradiating comprises irradiating a fixture in the disinfection area by the first wavelength.

30. The disinfection method of claim 24, wherein
the irradiating comprises irradiating a fixture in the disinfection area by the second wavelength.
